(19) United States
(12) Patent Application Publication
(10) Pub. No.: US 2011/0319786 A1
(43) Pub. Date: Dec. 29, 2011

(54) Apparatus and Method for Measuring Physiological Functions

(76) Inventors: Keith J. Rebello, Bethesda, MD (US); Joany Jackman, Walkersville, MD (US)

(21) Appl. No.: 12/951,600

(22) Filed: Nov. 22, 2010

Related U.S. Application Data
(60) Provisional application No. 61/262,944, filed on Nov. 20, 2009.

(51) Int. Cl. A61B 5/04; C23F 1/00; B29C 33/40; A61B 5/053

(52) U.S. Cl. 600/547; 600/382; 216/11; 264/224

(57) ABSTRACT

A sensor for monitoring a physiological function such as hydration including microelectrode arrays and electronics operatively connected and placed on an elastic strip, the assembled sensor being attached to a person in such a manner as to pinch and raise the skin under the elastic strip.

### CROSS-REFERENCE TO RELATED APPLICATIONS

This application relates to U.S. provisional application No. 61/262,944 filed on Nov. 20, 2009, which is incorporated herein by reference in its entirety.

### BACKGROUND OF THE INVENTION

The present invention relates to a sensor system and method for measuring a physiological function and, more specifically, to a microelectromechanical system (MEMS) hydration sensor.

Dehydration reduces both the mental and physical performance of an individual. If left untreated, dehydration can place the individual at risk of health problems and even death. Hydration monitoring has significant value in maximizing performance while also reducing health risks.

Whole body bioimpedance analysis (BIA) and bioimpedance spectroscopy (BIS) methods can be used to measure hydration. Bioimpedance was first used in the 60s to measure total water content. These techniques were refined in the 90s and BIA started to be used in the clinical environment. This measurement technique has advantages in that it is non-invasive, quick, and inexpensive but also has several limitations which make it impractical for measuring hydration in active participants.

The impedance of biological tissue is comprised of two components, resistance and reactance. The resistance component comes from the extracellular and intracellular conductive fluids, while the reactance component comes from cell membranes which act as capacitors. The reactance is frequency dependant. Typically a bipolar or tetrapolar measure is made where a voltage is applied to the body at multiple frequencies ranging from 1 kHz to 1.35 MHz. The resulting current is measured and impedance calculated.

At lower frequencies the cell membranes of the individual cells act as insulators and current cannot pass through. This results in the measurement of the extracellular fluid spaces (ECF) of the body. At high frequencies, the current penetrates the cell membrane giving a reading of both ECF and intracellular fluid (ICF) spaces.

Based on physiological models, equations have been developed to determine total body water, extracellular water, and intracellular water levels. While these equations have been used to measure hydration, they are not very accurate due to variations between person to person including tissue composition, ethnicity, gender, body shape and dimensions, etc.

Additionally, a strict set of standardized testing procedures must be followed for accurate readings. Subjects are required to lie down on a nonconductive mat. Individuals lie on their backs with their arms away from their sides and their legs slightly separated. All metal objects are removed. For a tetrapolar measurement, four large electrodes are attached to the subject at the following sites: wrist, hand, ankle, and foot. Wires are then clipped to each electrode.

The following procedures must also be followed:
cleaning of the skin where the electrodes contact with alcohol wipes;
accurate measurement of height and weight;
careful placement of gel electrodes to ensure proper position and full contact with skin;
minimization of time in recumbent position before measurements are made;
consistency in angle of abduction of limbs (ideal is)30-45° for recumbent measurements;
fasting for 4 hours prior to measurements;
controlled constant room temperature; and
avoidance of exercise for several hours prior to measurements.

Since current flows through a large part of the body it is susceptible to interference from the natural electrical signals in the body, which is why subjects must remain still during the measurement. This measurement system is not suitable for real time continuous monitoring of active individuals. Another disadvantage of surface electrodes is that a high current (800 uA) and high voltage must be utilized to decrease the instability of injected current related to the high impedance (10 000 O/cm2) of the skin. This also increases the size of the electrodes. BIA measurements are not recommended for those subjects who have a pacemaker or other electric stimulation devices.

In summary, current methods for assessing hydration status are invasive, expensive, slow, subjective or of limited accuracy. What is needed then is a low-cost (less than $250), small, fast response (less than one minute), non-invasive sensor for quickly and accurately providing a quantitative measure of the hydration status and rehydration needs of active individuals.

### SUMMARY OF THE INVENTION

Therefore, the present invention has been made in view of the above problems and constraints, and provides an apparatus and method to achieve the above objectives.

More specifically, the present invention is directed to an improved hydration sensor which uses MEMS technology to overcome the limitations of current hydration sensors. The invention is a cost effective disposable sensor which can provide hydration monitoring for the continuous monitoring of active individuals, but also has the flexibility to be used for a wide variety of other applications.

The invention is further directed to implementing a bioimpediance skin tenting test for measuring hydration, minimizing interference by using a localized measurement with improved electrodes utilizing microelectromechanical systems (MEMS) technology. Local BIA measurements have been shown to be an effective way to make measurements while removing traditional BIA's sensitivities to variations in body dimensions, ethnicity, and gender. MEMS probes for the measurement of bioimpedance signals have also been demonstrated.

The invention utilizes MEMS technology developed for the collection of skin tissue (see Rebello, K. J., MEMS Skin Therapies (INVITED), in Proceedings of the International Medical Devices Expo 2007, 2007: Burlington (hereinafter “Rebello”), which is incorporated herein by reference in its entirety). In this research flexible biocompatible arrays of microstructures were fabricated and tested, and successfully demonstrated their ability to penetrate and extract skin tissue from human subjects. The invention builds upon these structures to fabricate the necessary electrodes for bioimpedance measurements.

The electrical instrumentation circuitry for the measurements will be transferred and implemented in an application specific integrated circuit (ASIC) chip which would then be integrated into the sensor device.

The MEMS hydration sensor is non-invasive and provides a quantitative measure of hydration status in less than one minute. By using a batch fabricated MEMS technology many electrode patches can be made in parallel, greatly reducing cost. The disposable MEMS electrode patch is estimated to cost ˜$5. The electronic components (instrumentation amplifiers, op-amps, microcontrollers, etc.) used to measure the hydration signal are estimated to cost $250. The cost of the electronic components are further reduced by one to two orders of magnitude by consolidating them onto a single die in an ASIC chip which can then be integrated into the sensor device. This will allow for a completely disposable sensor device.

The invention is further directed to a sensor for monitoring a human physiological function comprising a strip of elastic material, the strip being attached to the skin; an electrode attached to a side of the strip being pressed against the skin, the electrode taking a bioimpedence measurement; and an electronics means operatively connected to the electrode, the electronic means receiving the bioimpedence measurement and determining a value for the monitored physiological function.

The invention is further directed to a method for monitoring a human physiological function comprising pressing an electrode against the skin using a strip of elastic material, the electrode being connected to an electronic means; taking a bioimpedence measurement using the electrode; transmitting the bioimpedence measurement to the electronic means; and determining a value for the monitored physiological function.

The invention is further directed to a method for making a micro piercing structure comprising etching the inverse of the structure into a substrate; applying a release layer to the etched substrate; pouring a biocompatible film into the etched substrate; curing the biocompatible film; and peeling the micro piercing structure from the etched substrate.

The invention is further directed to a method for making a micro piercing structure comprising fabricating a positive image of the micro piercing structure on a substrate; coating the substrate with a first release layer; casting an inverse mold of the micro piercing structure on the substrate using a polymer; curing the polymer inverse mold; peeling the polymer inverse mold off of the substrate; coating the polymer inverse mold with a second release layer; pouring a biocompatible film into the polymer inverse mold; curing the biocompatible film; and peeling the micro piercing structure from the polymer inverse mold.

### BRIEF DESCRIPTION OF THE DRAWINGS

The teachings of the present invention can be readily understood by considering the following detailed description in conjunction with the accompanying drawings, in which:

FIG. 1 illustrates the sensor of the invention and its use in practice.

FIG. 2 illustrates MEMS structures for skin therapies.

FIG. 3, comprising FIGS. 3A-3C, illustrates one method of the invention for fabricating flexible biocompatible micro piercing substrates.

FIG. 4, comprising FIGS. 4A-4F, illustrates a second method of the invention for fabricating flexible biocompatible micro piercing substrates.

FIG. 5 illustrates the test set up to test the electrodes of the invention.

FIG. 6 is a graph of hydrated tissue resistance vs. time at 1 Hz resulting from the tests using the test set up of FIG. 5.

FIG. 7 is a graph of hydrated tissue resistance vs. time at 100 Hz resulting from the tests using the test set up of FIG. 5.

FIG. 8 is a graph of impedance magnitude vs. frequency for various levels of hydration resulting from the tests using the test set up of FIG. 5.

### DETAILED DESCRIPTION

In the following discussion, numerous specific details are set forth to provide a thorough understanding of the present invention. However, those skilled in the art will appreciate that the present invention may be practiced without such specific details. In other instances, well-known elements have been illustrated in schematic or block diagram form in order not to obscure the present invention in unnecessary detail.

Reference will now be made in detail to the exemplary embodiments of the present invention, examples of which are illustrated in the accompanying drawings.

A skin tenting test is often used as a rough index of an individual's state of hydration by EMTs. When the skin and underlying subcutaneous tissue are pinched, raised up, and released, they return to a flat state without delay. When an individual is dehydrated the response becomes progressively slower. The invention provides for an improved skin tenting measurement by pinching the skin and using microscopic electrodes to take a bioimpedance measurement of the subcutaneous tissue.

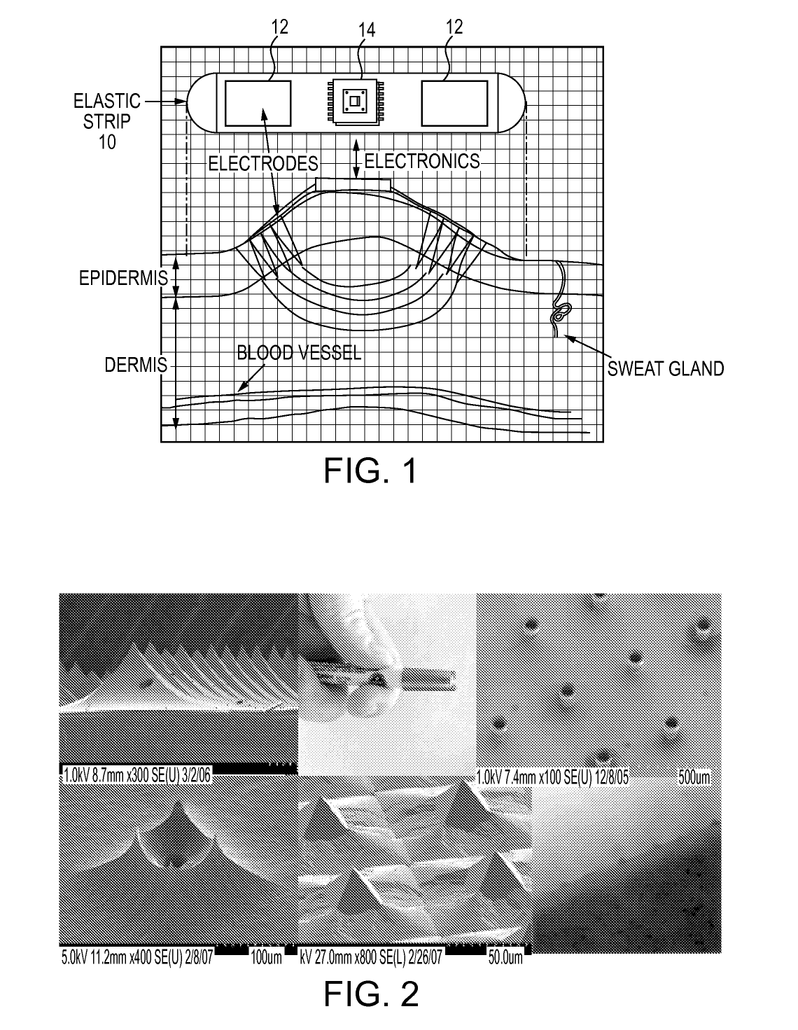

As shown in FIG. 1, the invention has an elastic strip 10 similar to an elastic band-aid or breathe right strip. This will provide the tension necessary to gather the skin. To overcome the high impedance of the skin and to eliminate interference from sweat secretion, microelectrode arrays 12 which penetrate into the epidermis will be used to take a low voltage measurement. The arrays are connected to electronics 14, preferably in an ASIC, which provides a quantitative value of hydration. A four point or two point impedance measurement at low and high frequencies would then be used to determine extracellular and total tissue water content. The user could be monitored continuously in real-time with an alarm sounding when dehydration becomes too great. By placing the invention in an out of the way location movement will not be restricted while also keeping electrical interference to a minimum.

This invention utilizes various micromechanical structures to pierce into the skin. As shown in FIG. 2, these structures have been fabricated on flexible substrates with biocompatible materials. These structures can be modified to serve as electrodes which will pierce into the outermost layer of skin (stratum corneum) improving contact and reducing the large surface areas needed with conventional electrodes. At the same time the electrodes are shallow enough to prevent contact with the nerves and blood vessels located below in the dermal skin layers, providing a non-invasive approach. In the fabrication of the electrode, only the tip of the electrode must be conductive so as not to encounter any skin surface effects. There are several techniques which have been reported in the literature to accomplish this (see Choi, S.-O., et al. An electrically active microneedle array for electroporation of skin for gene delivery. 2005. Seoul, South Korea: Institute of Electrical and Electronics Engineers Computer Society, Piscataway, N.J. 08855-1331, which is incorporated by reference herein in its entirety).

Applicants have developed processes to fabricate the structures on a flexible substrate. This will enable the MEMS electrode arrays to conform to any surface. One process is described in Rebello. Other processes are described in the two techniques below.

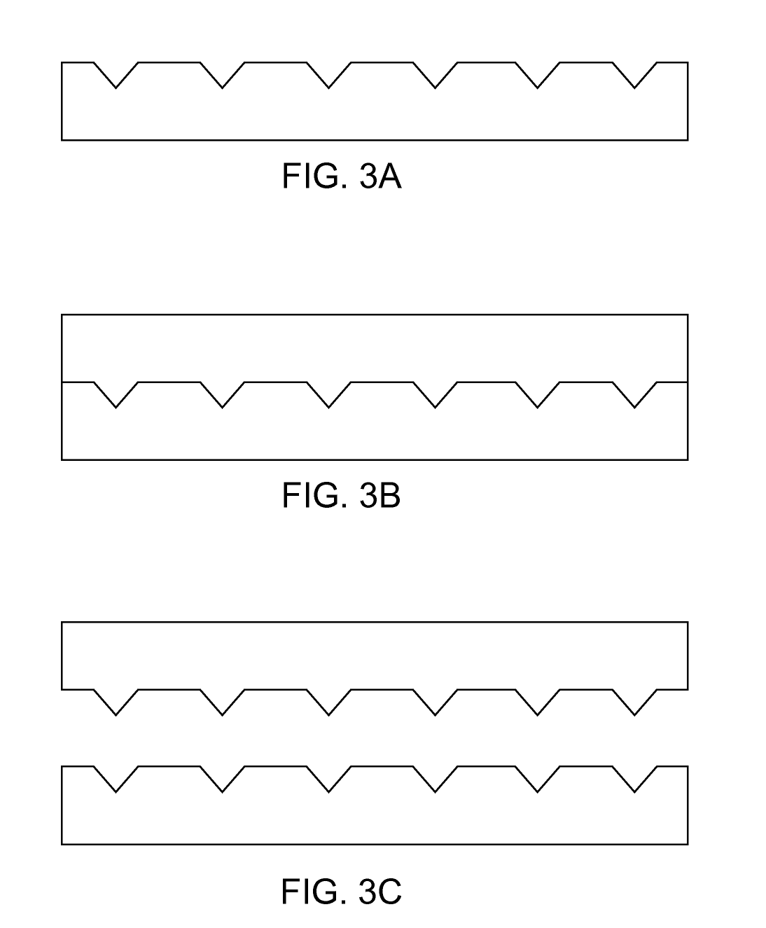

The first technique is a molding process where the inverse of the desired structures are etched into a substrate (FIG. 3A). Substrate materials and etch techniques can vary. Start with a silicon wafer and grow silicon nitride on the surface to serve as an etch mask for subsequent wet etching using KOH. The silicon wafers can be etched using other wet etchants such as TMAH or dry etch techniques such as RIE and DRIE. Next a release layer (fluoropolymer or silane) is applied to the wafer to prevent the bio compatible material from sticking to the mold. Next a biocompatible polymer film is poured into the mold, using, for example, urethane and PMMA materials (FIG. 3B). Once cured the micro piercing substrates can be peeled from the mold and the molds reused. (FIG. 3C).

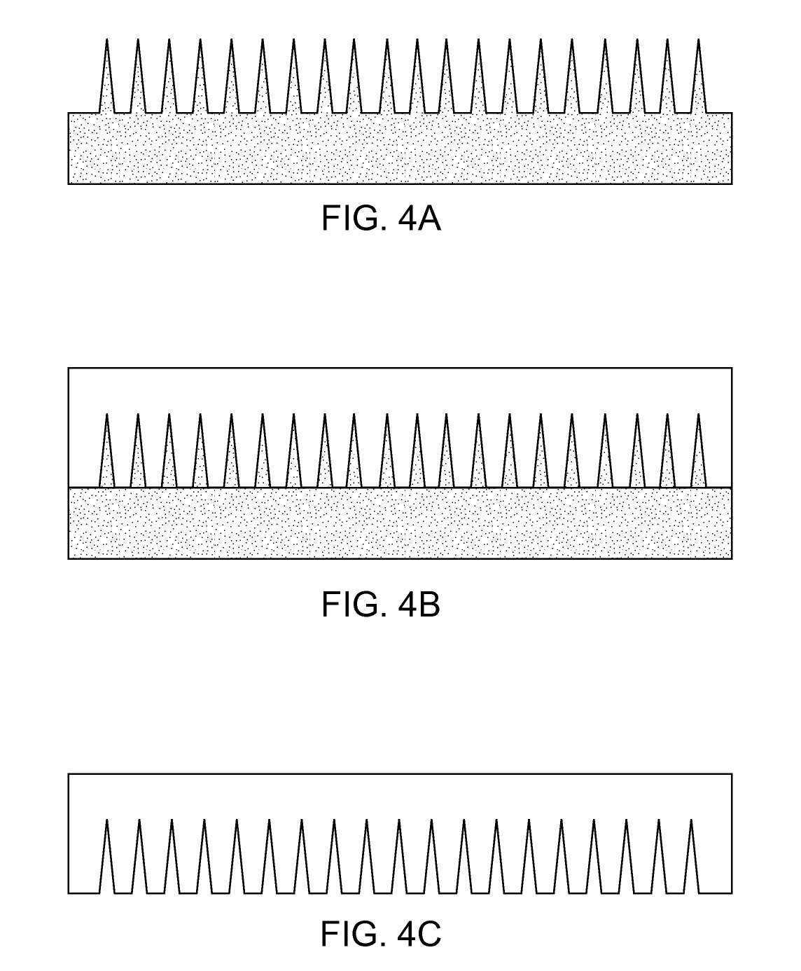

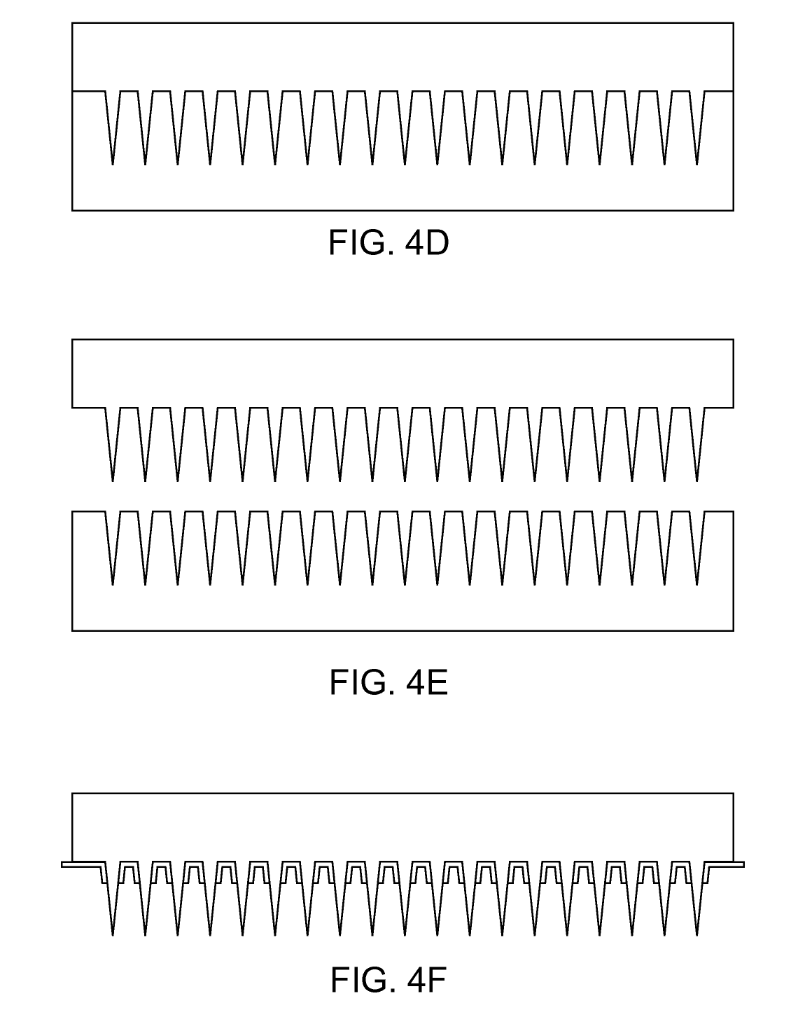

The second technique is useful when it is not possible to fabricate the inverse of the desired structures. In this case a positive image is fabricated using a substrate and etch/deposition technology of choice (FIG. 4A). In one case, a silicon wafer and KOH wet etchant were used along with corner compensation structures to achieve pyramid structures with very high aspect ratios.

Once the positive masters are made the molds are coated with a release layer (fluoropolymer or silane). Next an inverse mold is made by casting a polymer which can comprise a silicone and, more specifically, polydimethylsiloxane (PDMS) (FIG. 4B). Once cured the PDMS inverse mold is peeled off (FIG. 4C). A silane coating or other release layer is applied to the PDMS mold. Next this mold is cast with biocompatible urethane or PMMA materials (FIG. 4D). Once cured the PDMS mold is peeled off and can be reused, leaving the biocompatible substrate (FIG. 4E). Once fabricated the biocompatible polymers can be gold coated for electrical applications. In one embodiment (not in the embodiment tested as described below), the electrodes are pressed through a thin insulating material (such as kapton or another polymer) to prevent sweat or surface liquids from interfering with the measurement (FIG. 4F). Applicant then tested such fabricated electrodes as described below.

To mimic the impedance properties of hydrated tissue, Applicant formulated a stimulant based on a polymeric hydrogel. The hydrogel consisted of 25% poly (vinyl alcohol) (PVOH) by weight, and 25% 1× phosphate buffered saline (PBS) by weight. The PVOH was provided by Aldrich, and had a weight average molecular weight of 85,000-124,000 g/mol.

The concept behind the use of a hydrogel is that the intracellular matrix in the body typically consists of polymeric collagen which is swollen with water. The salt concentration and pH are nearly identical to 1×PBS. PBS is therefore commonly used in biological experiments.

To prepare the hydrogel, Applicant mixed 4 g PVOH with 12 g PBS. With its high molecular weight, PVOH does not readily dissolve in water, rather it swells to form a clear gel. This process was accelerated by heating at 100° C. and heating overnight.

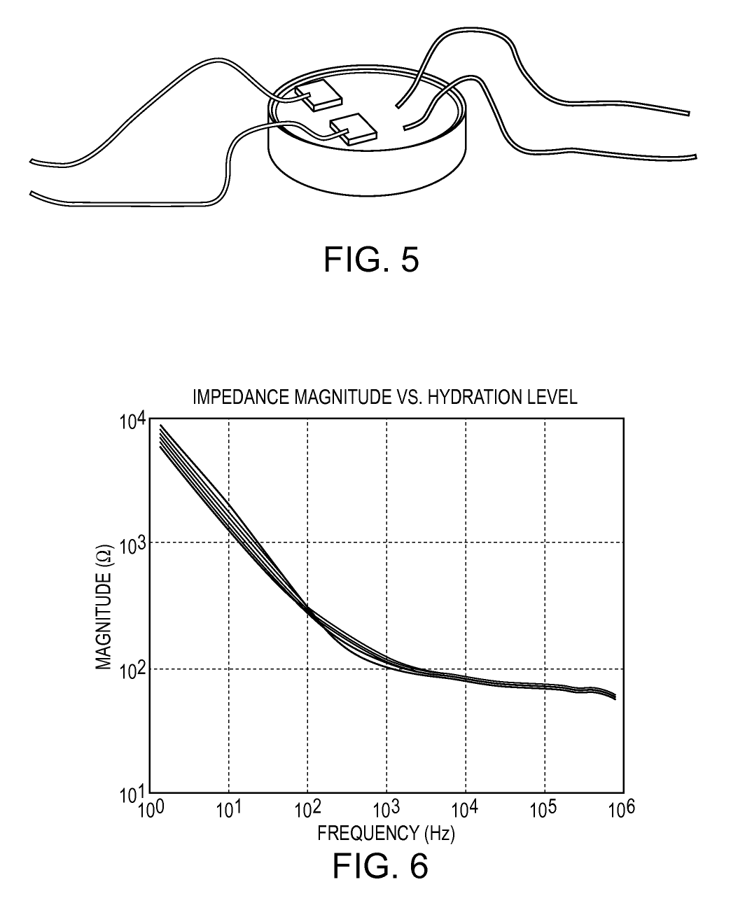

Once homogenized, the gel was cooled to room temperature, and placed in a small petri dish. Two gold electrodes were then embedded within the gel while spaced approximately 1 cm apart. Two platinum wires were also embedded 1 cm apart for comparison (FIG. 5).

Applicant then conducted impedance measurements once per hour, every hour for 12 hours. The impedance was measured as a function of time to evaluate whether the state of hydration could be monitored by measuring the real and imaginary components of the adhesion. The expectation is that the sodium phosphate will conduct through the hydrogel network with more ease at greater hydration levels. Despite the fact that the hydrogel was a tough solid, it was still 75% electrolyte at the start of the experiments. It was significantly drier after 12 hours.

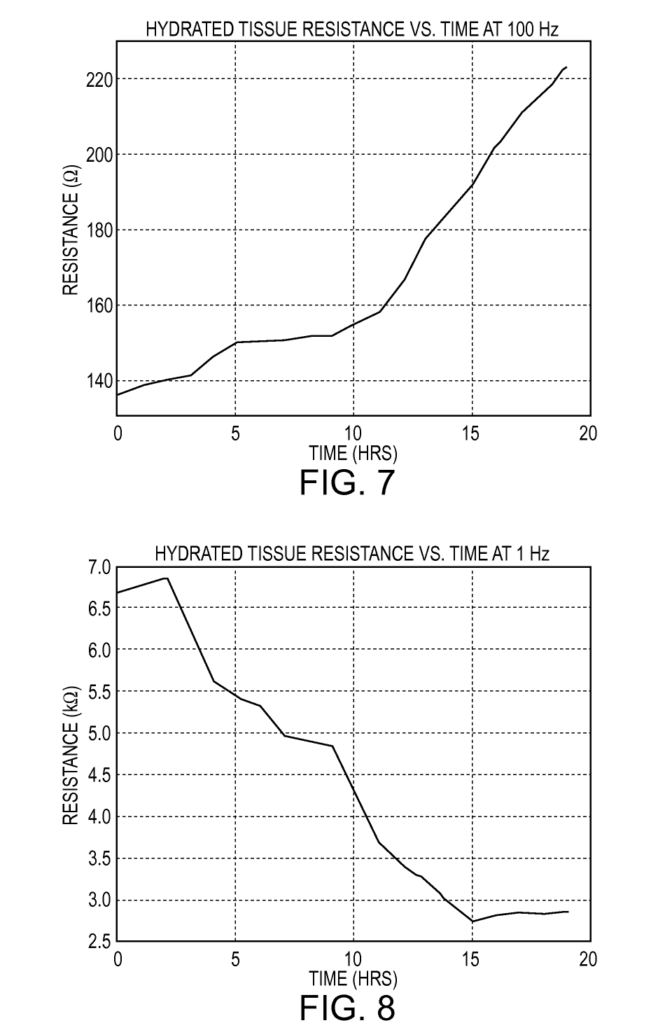

The hydrogel exhibited a consistent response with hydration level for its resistive component over specific measured frequencies. At 1 Hz and close to DC, its resistance was found to be inversely proportional to hydration level, with the equivalent extracellular fluid component dominant as is shown in FIG. 6. In addition, the material exhibited an increasing response at 100 Hz as is shown in FIG. 7. At these low frequencies, the material exhibited a highly capacitive component in its overall impedance, which is congruent with the model for intracellular fluid. At high frequency, both the equivalent intracellular and extracellular impedances dominated, reducing the overall impedance to the system through parallel combination and increasing the overall inductance in the material.

Interestingly, the magnitude and phase of the hydrogel were not linearly proportional with hydration level, as is shown in FIG. 8. It can also be inferred that the impedance model for the hydrogel is not entirely consistent with that of the intracellular and extracellular fluids due to the increased inductive component at high frequency. However, the hydrogel does properly model a lower impedance for the intracellular fluid and higher impedance for the extracellular fluid, as is predicted.

Preventing dehydration is a vital part of the wound healing process. The present invention could also be used to monitor a wound as it heals to make sure it is properly hydrated during the healing process. In addition the same electrode structures could be used actively, to drive therapeutic agents deeper into the wound by applying electric fields to the site. In this instance a/c voltage would be applied to two electrodes instead of measuring impedance.

It should be apparent to those skilled in the art that the present invention may be embodied in many other specific forms without departing from the spirit or scope of the invention. Therefore, the present examples and embodiments are to be considered as illustrative and not restrictive, and the invention is not to be limited to the details given herein, but may be modified within the scope of the appended claims.

What is claimed is:

1. A sensor for monitoring a human physiological function comprising:
a strip of elastic material, the strip being attached to the skin;
an electrode attached to a side of the strip being pressed against the skin, the electrode taking a bioimpedence measurement; and
an electronics means operatively connected to the electrode, the electronic means receiving the bioimpedence measurement and determining a value for the monitored physiological function.

2. The sensor as recited in claim 1, further comprising at least two electrodes, each electrode comprising a microelectrode array of microscopic electrodes for penetrating into the skin to take the bioimpedence measurement.

3. The sensor as recited in claim 2, wherein the strip when attached to the skin pinches the skin thereby raising the skin up under the strip.

4. The sensor as recited in claim 3, the monitored physiological function being hydration.

5. A method for monitoring a human physiological function comprising:
pressing an electrode against the skin using a strip of elastic material, the electrode being connected to an electronic means;
taking a bioimpedence measurement using the electrode;
transmitting the bioimpedence measurement to the electronic means; and
determining a value for the monitored physiological function.

6. The method as recited in claim 5, wherein the pressing the electrode step comprises pressing at least two electrodes against the skin.

7. The method as recited in claim 6, wherein each of the at least two electrodes comprises a microelectrode array of microscopic electrodes for penetrating into the skin to take the bioimpedence measurement.

8. The method as recited in claim 7, wherein the strip when attached to the skin pinches the skin thereby raising the skin up under the strip.

9. The method as recited in claim 8, wherein the monitored physiological function being hydration.

10. A method for making a micro piercing structure comprising:
etching the inverse of the structure into a substrate;
applying a release layer to the etched substrate;
pouring a biocompatible film into the etched substrate;
curing the biocompatible film; and
peeling the micro piercing structure from the etched substrate.

11. The method for making a micro piercing structure as recited in claim 10, wherein the etched substrate comprises a silicon wafer.

12. The method for making a micro piercing structure as recited in claim 10, wherein the biocompatible film comprises a polymer.

13. The method for making a micro piercing structure as recited in claim 10, wherein the release layer comprises one of either a fluoropolymer or a silane.

14. The method for making a micro piercing structure as recited in claim 10, further comprising growing silicon nitride on the surface of the substrate before the etching step, the silicon nitride serving as an etch mask.

15. A method for making a micro piercing structure comprising:
fabricating a positive image of the micro piercing structure on a substrate;
coating the substrate with a first release layer;
casting an inverse mold of the micro piercing structure on the substrate using a polymer;
curing the polymer inverse mold;
peeling the polymer inverse mold off of the substrate;
coating the polymer inverse mold with a second release layer;
pouring a biocompatible film into the polymer inverse mold;
curing the biocompatible film; and
peeling the micro piercing structure from the polymer inverse mold.

16. The method for making a micro piercing structure as recited in claim 15, the fabricating step comprising etching the substrate to form the positive image of the micro piercing structure.

17. The method for making a micro piercing structure as recited in claim 15, the fabricating step comprising depositing a material on the substrate to form the positive image of the micro piercing structure.

18. The method for making a micro piercing structure as recited in claim 15, wherein the substrate is a silicon wafer.

19. The method for making a micro piercing structure as recited in claim 15, wherein the biocompatible film comprises one of either urethane or PMMA.

20. The method for making a micro piercing structure as recited in claim 15, wherein the polymer comprises a silicone.

21. The method for making a micro piercing structure as recited in claim 20, wherein the silicone comprises polydimethylsiloxane.

22. The method for making a micro piercing structure as recited in claim 10 or 15, further comprising coating the micro piercing structure with gold to form an electrode.
